(19) United States
(12) Patent Application Publication
(10) Pub. No.: US 2015/0374330 A1
(43) Pub. Date: Dec. 31, 2015

(54) BLOOD VESSEL SEARCH DEVICE, ULTRASONIC MEASUREMENT APPARATUS, AND BLOOD VESSEL SEARCH METHOD

(71) Applicant: SEIKO EPSON CORPORATION, Tokyo (JP)

(72) Inventor: Kiyoaki MURAI, Matsumoto-shi (JP)

(21) Appl. No.: 14/753,336

(22) Filed: Jun. 29, 2015

(30) Foreign Application Priority Data
Jun. 30, 2014 (JP) 2014-133750

(51) Int. Cl. A61B 8/08; A61B 8/00

(52) U.S. Cl. CPC A61B 8/0891; A61B 8/5223; A61B 8/46

(57) ABSTRACT

An ultrasonic measurement control unit of an ultrasonic measurement apparatus generates received data by transmitting an ultrasonic wave to a blood vessel and receiving the ultrasonic wave reflected from the blood vessel. A blood vessel determination unit determines the blood vessel from the received data. A transmission direction setting unit sets the transmission direction of the ultrasonic wave for measuring the diameter of the blood vessel using the determination result of the blood vessel position. A blood vessel diameter measurement unit measures the diameter of the blood vessel by transmitting the ultrasonic wave in the transmission direction.

### BACKGROUND

1. Technical Field

The present invention relates to a blood vessel search device or the like that determines a blood vessel using an ultrasonic wave.

2. Related Art

As measurement methods using an ultrasonic wave, a linear scan to perform a scan by transmitting ultrasonic beams in parallel to each other and a sector scan to perform a scan by transmitting ultrasonic beams in a radial pattern are known. In addition, an oblique linear scan to transmit ultrasonic beams obliquely in the linear scan is also known (for example, refer to JP-A-6-114058).

In the linear scan, the arrangement range of ultrasonic transducers is the width of an observation region (scanning range). On the other hand, in the sector scan, the arrangement range of ultrasonic transducers is an observation region that spreads in a fan shape toward the depth direction. Accordingly, the width of the observation region in the sector scan is larger than that in the linear scan. For this reason, for the observation of a deep part, the sector scan with a large deep field width is preferred. In the case of the sector scan, however, the near field width is small. Therefore, for the observation of a shallow part located about 100 millimeters away from the skin surface, the linear scan is preferred.

When the target of ultrasonic measurement is a blood vessel, a change in the blood vessel diameter due to pulsation is measured, in many cases, by performing tracking to track the position of a blood vessel wall using a received signal related to the scanning line passing through the center of the blood vessel. For example, phase difference tracking is a technique for tracking the displacement between different frames in the same scanning line in a time series, that is, a technique for tracking the displacement of the blood vessel wall in the transmission direction of the ultrasonic beam. In the phase difference tracking, it is necessary to set the scanning line used for tracking so as to pass through the center of the blood vessel.

In the known tracking methods, however, it has been common to fix the initially set scanning line and track the displacement of the blood vessel wall in the scanning line. For this reason, if the blood vessel is displaced, the scanning line does not pass through the center of the blood vessel. This has caused a situation where it is not possible to measure the correct vessel diameter. For example, when performing continuous ultrasonic measurement for the carotid artery, the position of the blood vessel is shifted by the rotation of the head, contraction of neck muscles, or the like.

### SUMMARY

An advantage of some aspects of the invention is to provide a technique for setting the transmission direction of an ultrasonic wave according to the position of a blood vessel so that the blood vessel diameter can be accurately measured.

A first aspect of the invention is directed to a blood vessel search device including: a received data calculation unit that calculates received data by transmitting an ultrasonic wave to a blood vessel and receiving the ultrasonic wave reflected from the blood vessel; a blood vessel determination unit that determines the blood vessel based on a calculation result of the received data calculation unit; a transmission direction setting unit that sets a transmission direction of the ultrasonic wave for measuring a diameter of the blood vessel using a determination result of the blood vessel determination unit; and a blood vessel diameter measurement unit that measures the diameter of the blood vessel by transmitting the ultrasonic wave in the transmission direction.

As the second aspect of the invention, the first aspect of the invention may be configured as a blood vessel search method including: calculating received data by transmitting an ultrasonic wave to a blood vessel and receiving the ultrasonic wave reflected from the blood vessel; determining the blood vessel based on a calculation result of the received data; and setting a transmission direction of the ultrasonic wave for measuring information of the blood vessel using the determination result.

According to the first aspect and the like of the invention, the blood vessel is determined based on the received data of the ultrasonic wave reflected from the blood vessel, and the transmission direction of the ultrasonic wave for measuring the information of the blood vessel is set using the determination result. In this case, since it is possible to set the ultrasonic wave transmission direction suitable for the position of the blood vessel, it is possible to accurately measure the information of the blood vessel. For example, it is possible to use a method of setting the transmission direction so as to follow the displacement of the blood vessel when necessary.

As a second aspect of the invention, the blood vessel search device according to the first aspect of the invention may be configured such that the received data calculation unit calculates the received data by transmitting the ultrasonic wave to the blood vessel in a plurality of the transmission directions and receiving the ultrasonic wave that is reflected.

According to the second aspect of the invention, received data is calculated by transmitting the ultrasonic wave in a plurality of transmission directions. That is, it is possible to widen the observation region by the ultrasonic wave.

As a third aspect of the invention, the blood vessel search device according to the first or second aspect of the invention may be configured such that the transmission direction setting unit sets the transmission direction such that a center of the blood vessel is included in a determination result of the blood vessel determination unit.

According to the third aspect of the invention, the ultrasonic wave transmission direction is set such that the center of the blood vessel is included in the determination result of the blood vessel. Therefore, a range including the center of the blood vessel can be set as an observation region.

As a fourth aspect of the invention, the blood vessel search device according to the third aspect of the invention may be configured such that the transmission direction setting unit sets the transmission direction to a direction passing through the center of the blood vessel from a part of a transmission unit of the ultrasonic wave.

According to the fourth aspect of the invention, the ultrasonic wave transmission direction is set so as to pass through the center of the blood vessel from a part of the transmission unit. Therefore, for example, if a part of the transmission unit is the center of the transmission unit, the blood vessel can be positioned at the center of the observation region in the width direction.

As a fifth aspect of the invention, an ultrasonic measurement apparatus including: the blood vessel search device according to any one of the first to fourth aspects of the invention; and a blood vessel information measurement unit that measures information of the blood vessel using the calculation result of the received data calculation unit in the transmission direction set by the transmission direction setting unit may be configured.

According to the fifth aspect of the invention, the information of the blood vessel is measured using the calculation result of the received data in the set transmission direction.

As a sixth aspect of the invention, the ultrasonic measurement apparatus according to the fifth aspect of the invention may be configured such that the blood vessel information measurement unit measures a diameter of the blood vessel using the calculation result in a transmission direction passing through a center of the blood vessel.

According to the sixth aspect of the invention, the blood vessel diameter is measured using the calculation result of the received data in the transmission direction passing through the center of the blood vessel. Therefore, it is possible to measure the diameter as a blood vessel diameter.

As a seventh aspect of the invention, the ultrasonic measurement apparatus according to the fifth or sixth aspect of the invention may be configured such that measurement of information of the blood vessel by the blood vessel information measurement unit is continued by executing the blood vessel determination of the blood vessel determination unit and the transmission direction setting of the transmission direction setting unit so as to follow displacement of the blood vessel.

According to the seventh aspect of the invention, the determination of the blood vessel and the setting of the transmission direction of the ultrasonic wave according to the determined blood vessel are executed so as to follow the displacement of the blood vessel. Therefore, it is possible to continuously measure the information of the blood vessel so as to follow the displacement of the blood vessel.

As an eighth aspect of the invention, the ultrasonic measurement apparatus according to any one of the fifth to seventh aspects of the invention may be configured to further include an ultrasonic wave output change unit that changes an output intensity of the ultrasonic wave according to a distance change from the transmission unit to the blood vessel due to displacement of the blood vessel.

According to the eighth aspect of the invention, the output intensity of the ultrasonic wave is changed according to the distance change from the transmission unit to the blood vessel. For example, by making the output intensity proportional to the distance, it is possible to perform ultrasonic measurement in a state where the received signal strength of the reflected wave is stable, regardless of the displacement of the blood vessel.

### BRIEF DESCRIPTION OF THE DRAWINGS

The invention will be described with reference to the accompanying drawings, wherein like numbers reference like elements.

FIG. 1 is a diagram showing the system configuration of an ultrasonic measurement apparatus.

FIGS. 2A and 2B are diagrams for explaining the transmission direction of the ultrasonic wave.

FIG. 3 is a diagram for explaining blood vessel determination.

FIGS. 4A and 4B are diagrams for explaining the setting of the transmission direction of the ultrasonic wave.

FIGS. 5A to 5C are diagrams for explaining an example of the received signal.

FIG. 6 is a diagram for explaining the determination of a scanning line passing through the center of the blood vessel.

FIGS. 7A and 7B are diagrams for explaining the measurement of the blood vessel diameter.

FIG. 8 is a diagram showing the functional configuration of the ultrasonic measurement apparatus.

FIG. 9 is a flowchart of the ultrasonic measurement process.

### DESCRIPTION OF EXEMPLARY EMBODIMENTS

### System Configuration

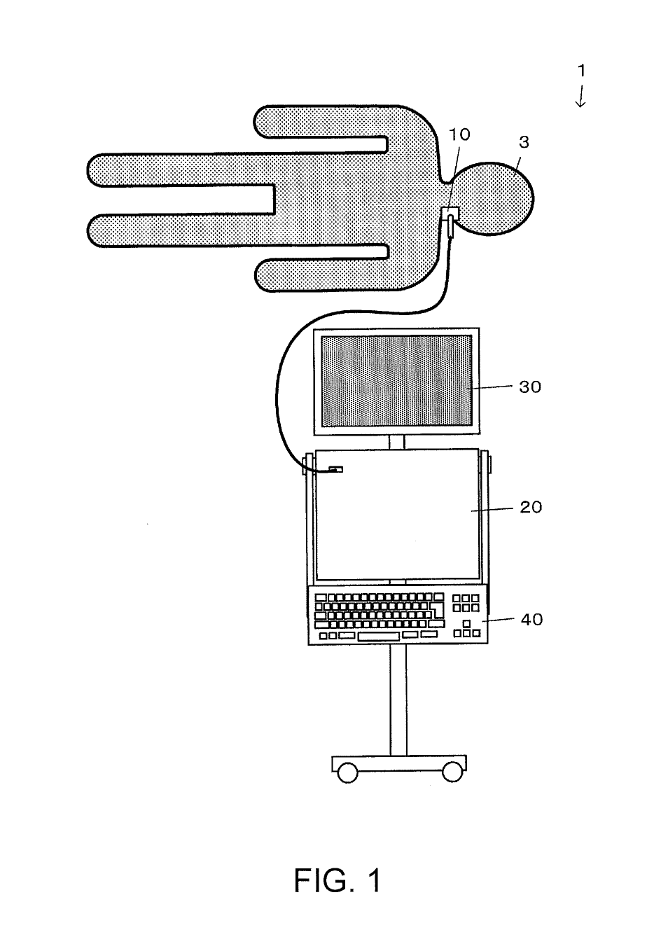

FIG. 1 is a diagram showing the system configuration of an ultrasonic measurement apparatus 1 according to the present embodiment. The ultrasonic measurement apparatus 1 is an apparatus for measuring the biological information of a subject 3 in a non-invasive way using an ultrasonic wave, and is also a blood vessel search device. In the present embodiment, vascular system function information, such as blood pressure or intima-media thickness (IMT) relevant to the carotid artery, is measured as a piece of biological information. The ultrasonic measurement apparatus 1 includes an ultrasonic probe 10, a main body unit 20, a video monitor 30, and a keyboard 40.

The ultrasonic probe 10 includes a plurality of ultrasonic transducers. Each ultrasonic transducer is a thin-film piezoelectric ultrasonic transducer that transmits an ultrasonic wave for measurement having a frequency of, for example, several MHz to tens of MHz and converts the reflected wave (ultrasonic echo) of the ultrasonic wave from the subject 3 into an electrical signal. The ultrasonic probe 10 outputs the received signal to the main body unit 20. The ultrasonic probe 10 is a thin planar pad type probe that can be attached to the neck or the like of the subject 3, and is used in a state of being attached and fixed to the neck of the subject 3. In addition, the fixing position of the ultrasonic probe 10 is not limited to the neck for the measurement of the carotid artery, and positions for the measurement of other arteries, such as a wrist for the measurement of the radial artery, are also possible.

The main body unit 20 is realized by various microprocessors such as a central processing unit (CPU), a graphics processing unit (GPU), and a digital signal processor (DSP), various IC memories such as an application specific integrated circuit (ASIC), an electronic circuit, a VRAM, a random access memory (RAM), and a read only memory (ROM), information storage media such as a hard disk, an interface IC or a connection terminal to realize the transmission and reception of data from the outside, a power supply circuit, and the like.

The main body unit 20 is wired with the ultrasonic probe 10, and generates received data of the reflected wave of the ultrasonic wave by performing ultrasonic measurement using the ultrasonic probe 10, calculates vascular system function information using the received data, and performs sequentially updated display of the calculation result on the video monitor 30.

The received data includes not only the received signal itself, which is data, such as temporal changes or position information of the in vivo structure of the subject 3, but also an image of each mode, such as a so-called A mode, B mode, M mode, and color Doppler. Measurement using an ultrasonic wave is repeatedly performed at predetermined periods. The measurement unit is referred to as a “frame”.

The video monitor 30 is an image display device, and is realized by a flat panel display or a touch panel display. Optionally, a speaker may be built into the video monitor 30.

The keyboard 40 is means used when the operator inputs various operations. In the example shown in FIG. 1, the keyboard is pivotably supported by the swing arm, and can be used by being moved to the front when necessary. However, the keyboard 40 may be formed integrally with the main body unit 20, or the video monitor 30 may also be made to serve as the keyboard 40 by adopting a touch panel type video monitor. In addition, it is also possible to add other operation input devices, such as a mouse and a track pad.

### Principle

### (A) Oblique Linear Scan

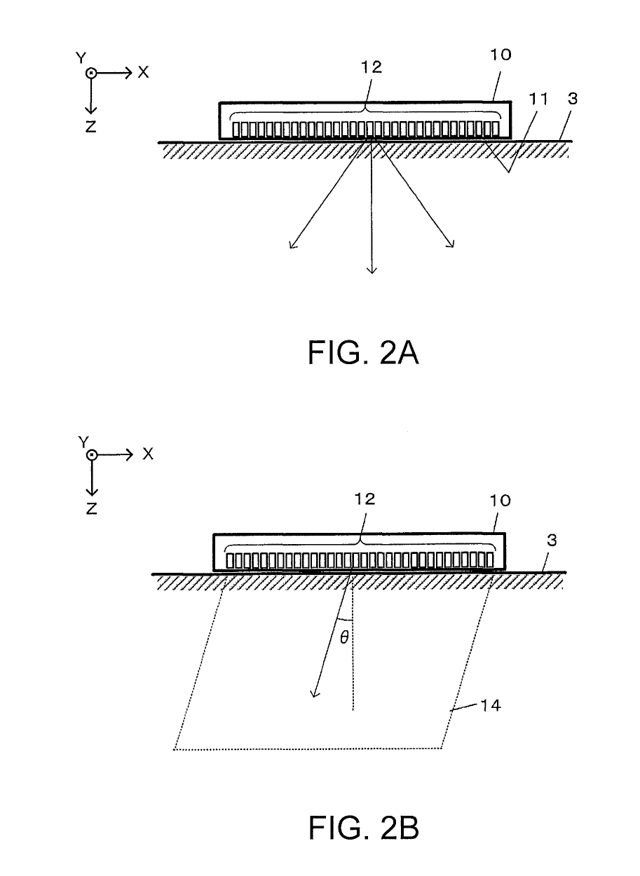

FIGS. 2A and 2B are diagrams schematically showing a state of performing ultrasonic measurement using the ultrasonic probe 10. The ultrasonic probe 10 is used in a state where a transmission surface 11 is attached to the body surface of the subject 3. On the transmission surface 11 side of the ultrasonic probe 10, a plurality of ultrasonic transducers 12 are provided so as to be arranged at equal distances therebetween in a row.

As shown in FIG. 2A, each of the ultrasonic transducers 12 transmits an ultrasonic beam. However, it is possible to change the transmission direction (focusing direction) by transmission focus control. In the present embodiment, explanation will be given with the coordinates of three axes perpendicular to each other that are based on the assumption that a normal direction with respect to the transmission surface 11 is a Z axis, the arrangement direction of the ultrasonic transducers 12 that is parallel to the transmission surface 11 is an X axis, and a direction that is parallel to the transmission surface 11 and is perpendicular to the X axis is a Y axis. The ultrasonic beam can be transmitted in a designated direction that is parallel to the X-Z plane and has an arbitrary scanning angle θ around the Y axis.

In addition, the ultrasonic beam can be transmitted using K (N≦K≦1: for example, 32) ultrasonic transducers 12 of N (for example, 256) ultrasonic transducers 12 arranged in a row. By the transmission focusing control to adjust the transmission timing of the ultrasonic wave from each ultrasonic transducer 12, it is possible to control the transmission direction (focusing direction) or the focusing position of the ultrasonic beam. For the simplicity of explanation, the following explanation will be given on the assumption that the ultrasonic beam is transmitted from one ultrasonic transducer 12 located at the center among the K ultrasonic transducers 12. However, this does not intend to exclude a case of transmitting one ultrasonic beam using the K ultrasonic transducers 12 adjacent to each other. Hereinafter, one transmission of the ultrasonic beam and one reception of the reflected wave will be appropriately referred to as “scanning”, the ultrasonic beam (or the ultrasonic transducer 12 that transmits the ultrasonic beam) will be appropriately referred to as a “scanning line”, and the transmission direction of the ultrasonic beam will be appropriately referred to as a “scanning angle”.

As shown in FIG. 2B, the transmission direction of the ultrasonic beam from the ultrasonic transducer 12 is expressed by the angle θ with respect to the Z axis (normal direction with respect to the transmission surface 11) on the X-Z plane. That is, a direction along the Z axis (normal direction with respect to the transmission surface 11) is assumed to be the transmission direction θ=0. In addition, in FIG. 2B, it is assumed that a clockwise direction with respect to the Z axis is a negative value (transmission direction θ<0) and a counterclockwise direction with respect to the Z axis is a positive value (transmission direction θ>0). In addition, due to the limitations on the structure of the ultrasonic transducer 12, the transmission direction of the ultrasonic beam can be changed in a range from a minimum transmission direction θmin (<0) to a maximum transmission direction θmax (>0).

In the present embodiment, it is assumed that the transmission direction θ of each ultrasonic transducer 12 in one transmission and reception of the ultrasonic wave is the same. That is, in one transmission and reception of the ultrasonic wave, ultrasonic beams transmitted from the respective ultrasonic transducers 12 are parallel. Accordingly, when transmitting ultrasonic beams from all of the ultrasonic transducers 12, an observation region 14 is a parallelogram region. The observation region 14 is changed by changing the transmission direction θ of the ultrasonic wave. Such control of the transmission direction of the ultrasonic wave is referred to as an oblique linear scan.

### (B) Blood Vessel Determination

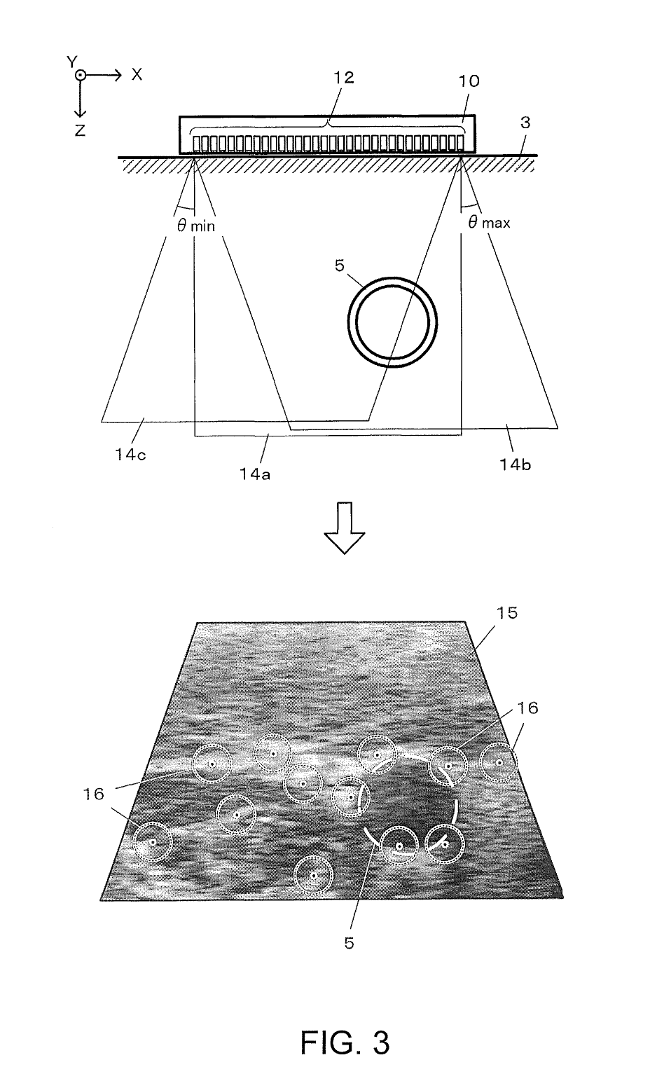

As shown in FIG. 3, by combining a plurality of B-mode images having different ultrasonic wave transmission directions θ by an oblique linear scan, a B-mode image of the maximum observation region, which is the maximum region that can be observed by the ultrasonic probe 10, is generated as wide-angle received data. Here, the B-mode image of the maximum observation region is received data of a wide angle. Specifically, a B-mode image 15 of the maximum observation region is generated by combining three B-mode images of a B-mode image of an observation region 14a when the transmission direction θ is set to “0”, a B-mode image of an observation region 14b when the transmission direction θ is set to the maximum transmission direction θmax, and a B-mode image of an observation region 14c when the transmission direction θ is set to the minimum transmission direction θmin. The maximum observation region is a trapezoidal region.

Blood vessel determination is performed in the B-mode image 15. For the blood vessel determination based on the B-mode image, any technique can be used. For example, as shown in FIG. 3, a feature point 16 in the B-mode image 15 is extracted. The feature point is a point that can be noticeably observed in the image. The reflectance of the ultrasonic wave is high at the change position of the medium (boundary of the medium), and a position of high reflectance in the B-mode image is expressed with high brightness. For this reason, in the B-mode image, a number of feature points appear not only in the blood vessel wall but also in portions where brightness changes occur, such as muscles, tendons, and fat. However, since the ultrasonic wave is transmitted through the blood, with little reflection, inside the blood vessel, few feature points appear inside the blood vessel. Therefore, by detecting a region corresponding to the inside of the blood vessel, it is possible to determine the blood vessel, that is, determine the position of the blood vessel in the B-mode image. Although the number of feature points 16 is intentionally set to a small number in FIG. 3, it is possible to extract a larger number of feature points 16 in practice.

As shown in FIG. 3, when a blood vessel 5 is located in the maximum observation region, the feature points 16 are distributed in the B-mode image 15, which is a cross-sectional view of the blood vessel 5 in the short-axis direction, such that an approximately circular shape corresponding to the cross-sectional shape of the blood vessel 5 in the short-axis direction is formed at a position corresponding to the blood vessel 5. Therefore, in the B-mode image 15, the distribution of approximately circular feature points is determined as the position of the blood vessel wall, and the center of the approximately circular shape is determined to be the position of the blood vessel. The center position of the blood vessel 5 can be calculated as the position coordinates on the X-Z plane.

### (C) Setting of Transmission Direction (Scanning Angle) of Ultrasonic Wave

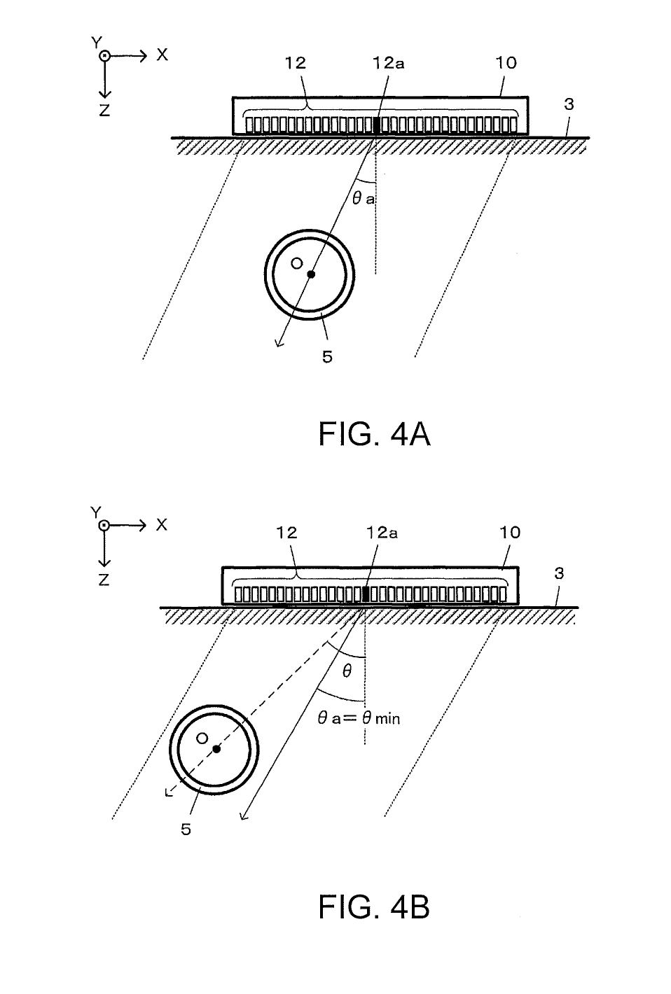

Based on the determined blood vessel, the transmission direction of the ultrasonic wave for the measurement of a blood vessel diameter is set. FIGS. 4A and 4B are diagrams for explaining the setting of the transmission direction of the ultrasonic wave. As shown in FIG. 4A, a transmission direction θa is set such that the ultrasonic beam transmitted from a central ultrasonic transducer 12a (that is, the center of the ultrasonic wave transmission unit), among the plurality of ultrasonic transducers 12 arranged in a row, passes through the center O of the blood vessel 5 to be measured. Accordingly, the blood vessel 5 to be measured is located at the approximate center in the X-axis direction of the observation region (column direction of the ultrasonic transducers 12).

The transmission direction θ of the ultrasonic transducer 12 can be set in the range from the minimum transmission direction θmin to the maximum transmission direction θmax. Therefore, as shown in FIG. 4B, when the transmission direction θ of the ultrasonic transducer 12a passing through the center O of the blood vessel 5 to be measured is outside of the range that can be set, the transmission direction θ is set so as to fall within the range that can be set. That is, the transmission direction θa is set to the minimum transmission direction θmin when the transmission direction θ is less than the minimum transmission direction θmin, and the transmission direction θa is set to the maximum transmission direction θmax when the transmission direction θ exceeds the maximum transmission direction θmax. In the example shown in FIG. 4B, since the transmission direction θ of the ultrasonic transducer 12a passing through the center O of the blood vessel 5 to be measured is less than the minimum transmission direction θmin, the transmission direction θa is set to the minimum transmission direction θmin.

### (D) Determination of Center Scanning Line

After setting the transmission direction θa of the ultrasonic wave, the ultrasonic transducer 12 used for the measurement of the blood vessel diameter is determined. In the present embodiment, the ultrasonic transducer 12 with an ultrasonic beam transmission direction passing through the center of the blood vessel is used for the measurement of the blood vessel diameter. This ultrasonic transducer 12 is referred to as a “center scanning line”.

Blood vessels largely contract and expand periodically due to the beating of the heart, but the movement of other biological tissues around the blood vessels is small compared with the movement of the blood vessels. Based on this finding, the center scanning line is determined. That is, the blood vessel repeats contraction and expansion approximately isotropically due to the beating of the heart. Therefore, in the ultrasonic measurement, it is possible to receive a stronger reflected wave as the wall portion is more perpendicular to the transmission direction of the ultrasonic wave, but the strength of the reflected wave that can be received becomes lower as the wall portion is more parallel to the transmission direction.

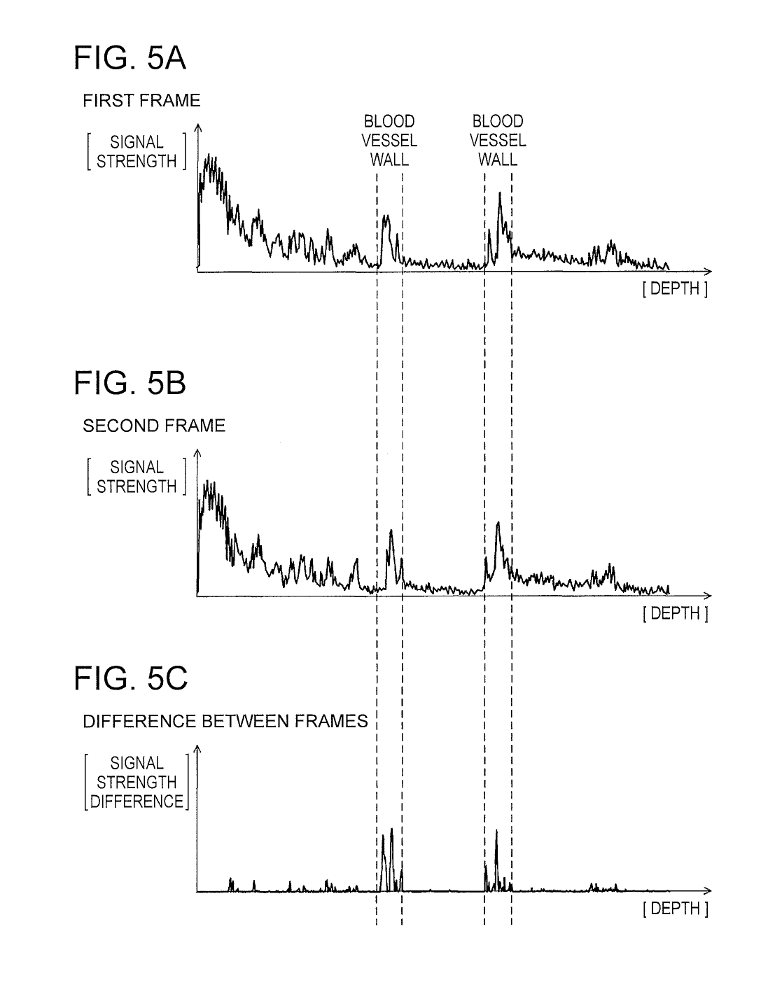

FIGS. 5A to 5C are diagrams for explaining the determination of the center scanning line, and show an example of the received signal of the reflected wave by the ultrasonic transducer 12 with an ultrasonic beam transmission direction passing through the center of the blood vessel. FIG. 5A is a graph of “depth-signal strength” showing the result (one scan) of ultrasonic measurement in the first frame of the measurement period, and FIG. 5B is a graph of “depth-signal strength” showing the result of ultrasonic measurement in the next second frame. FIG. 5C is a graph of “signal strength difference between frames” obtained by calculating the difference between the signal strength in the first frame and the signal strength in the second frame for each depth.

If there is a blood vessel in the transmission direction of the ultrasonic beam, a strong reflected wave relevant to the blood vessel wall is detected. Also in FIGS. 5A and 5B, two strong reflected wave peaks that can be clearly distinguished appear at positions deeper than the reflected wave group near the body surface. In addition, as shown in FIG. 5C, the movement of the blood vessel wall becomes clear if the signal strength difference between the first and second frames is calculated for each depth.

As is apparent from the graph in FIG. 5C, a slight signal strength difference occurs even in portions other than the blood vessel because body tissues other than the blood vessel are also slightly moved due to the influence of the pulsation of the blood vessel or the like. However, a value that is as large as the value for the blood vessel wall is not detected. Even more, such a peak is not seen in the signal strength difference graph of the reflected wave signal in an ultrasonic transducer with no blood vessels in the transmission direction of the ultrasonic beam. That is, it can be said that the movement of the blood vessel wall due to pulsation appears in a change in the signal strength between frames having a time difference therebetween.

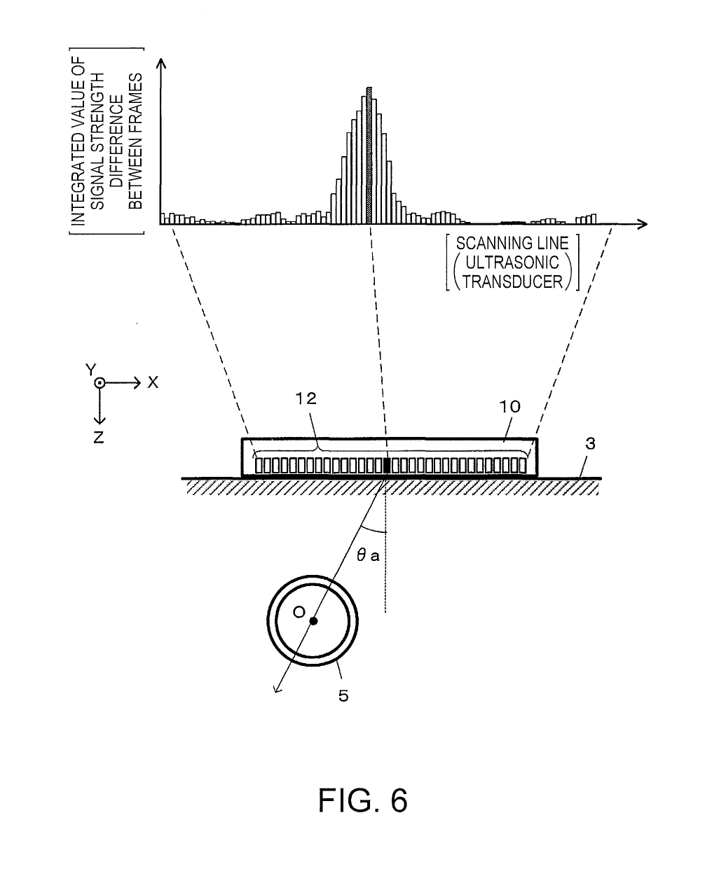

FIG. 6 is a histogram obtained by integrating the signal strength difference between consecutive frames for each ultrasonic transducer 12. The horizontal axis indicates the arrangement order (scanning direction) of the ultrasonic transducers 12, and the vertical axis indicates an integrated value of the signal strength difference in each ultrasonic transducer 12. That is, the integrated value is a value obtained by calculating the sum of signal strength differences between two frames at all depths obtained as the “graph of signal strength difference between frames” shown in FIG. 5C, for each ultrasonic transducer 12, and further integrating the total value over a predetermined period of time (for example, at least 1 to several beats of a cardiac period).

For the sum of the signal strength differences obtained from the ultrasonic measurement for two consecutive frames, the ultrasonic transducer 12 with an ultrasonic beam transmission direction passing through the blood vessel shows a larger value than the ultrasonic transducer 12 with an ultrasonic beam transmission direction not passing through the blood vessel. In addition, for the sum of the signal strength differences obtained from the ultrasonic measurement for two consecutive frames, the ultrasonic transducers 12 with a transmission direction passing through a position closer to the center of the blood vessel 5 shows a larger value. Accordingly, the ultrasonic transducer 12 with a peak integrated value of signal strength differences is determined to be the ultrasonic transducer 12 with an ultrasonic beam transmission direction passing through the center of the blood vessel, that is, determined to be the center scanning line. The method of determining the center scanning line is an example, and the invention is not limited thereto.

### (E) Measurement of Blood Vessel Diameter

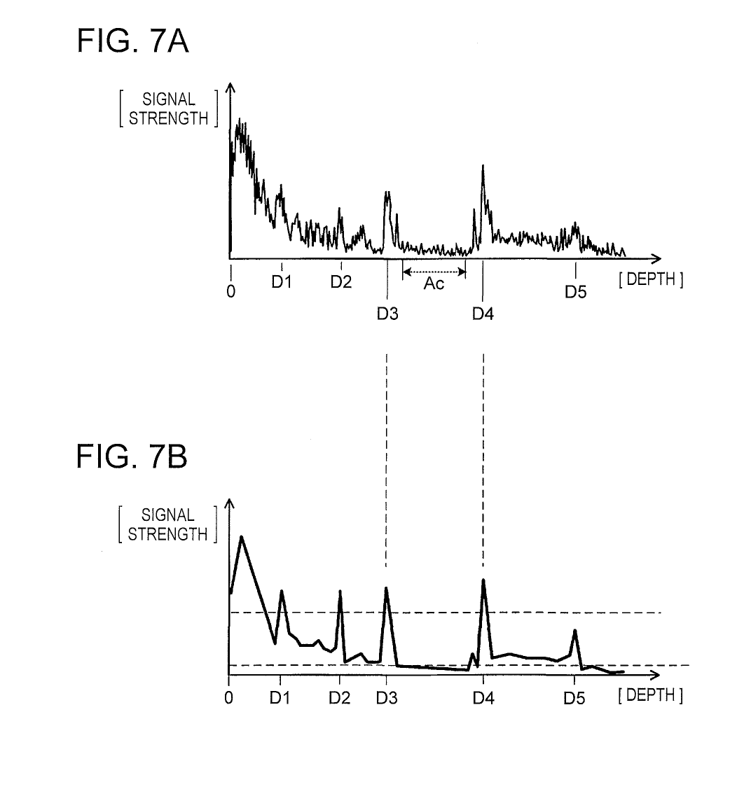

After determining the center scanning line, the blood vessel diameter is measured by ultrasonic measurement using the center scanning line. First, a blood vessel wall is determined. FIGS. 7A and 7B are diagrams for explaining the determination of a blood vessel wall. FIG. 7A is a signal strength graph of the received signal of the reflected wave in the center scanning line, and FIG. 7B is a graph obtained by smoothing changes in the signal strength in the graph of FIG. 7A more clearly. From the received signal, two peaks, which are equal to or greater than a predetermined signal strength and between which a signal strength difference is equal to or less than a predetermined low strength indicating the blood, is determined as a blood vessel wall.

Then, a signal portion relevant to the two peaks determined to be the blood vessel wall is set as a tracking region, the position of the tracking region is tracked over a plurality of consecutive frames using the received signal of the reflected wave based on the center scanning line, and displacement in a direction along the center scanning line of the blood vessel wall is tracked. As a result, the blood vessel diameter is calculated.

### Functional Configuration

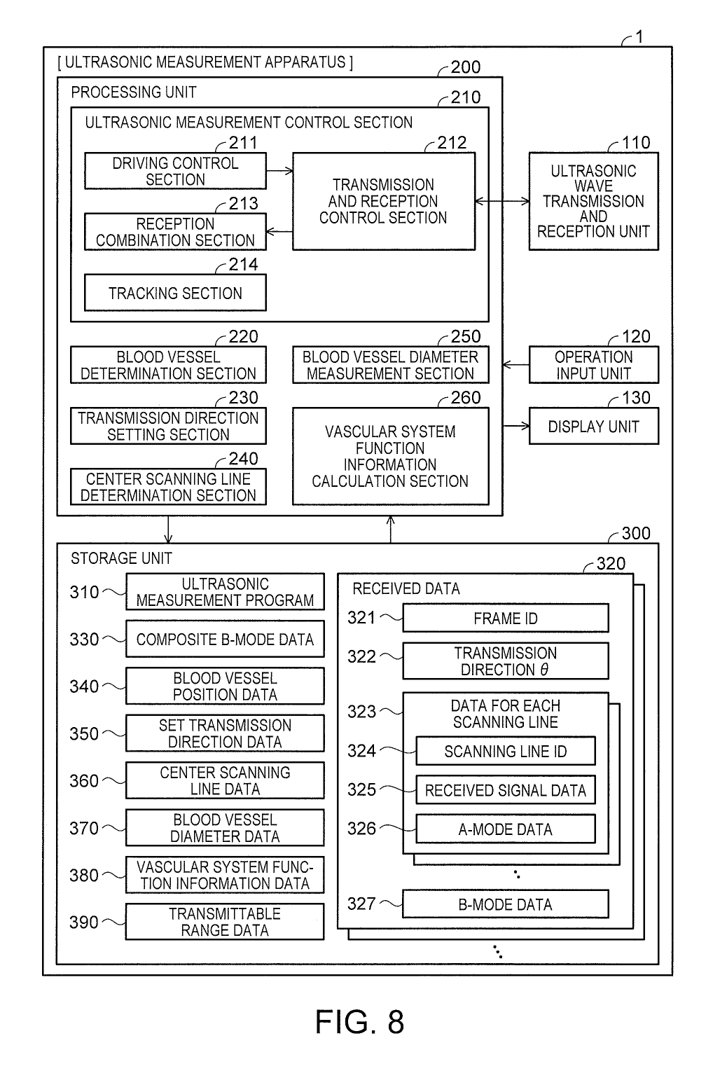

FIG. 8 is a block diagram showing the functional configuration of the ultrasonic measurement apparatus 1. As shown in FIG. 8, the ultrasonic measurement apparatus 1 includes an ultrasonic wave transmission and reception unit 110, an operation input unit 120, a display unit 130, a processing unit 200, and a storage unit 300.

The ultrasonic wave transmission and reception unit 110 includes a plurality of ultrasonic transducers for transmitting and receiving an ultrasonic wave that are arranged in a row. Each ultrasonic transducer transmits an ultrasonic wave corresponding to the pulse voltage input from the processing unit 200 and receives a reflected wave of the ultrasonic wave, converts the reflected wave into a reflected wave signal that is an electrical signal, and outputs the reflected wave signal to the processing unit 200. The ultrasonic probe 10 shown in FIG. 1 corresponds to the ultrasonic wave transmission and reception unit 110.

The operation input unit 120 receives various kinds of operation input from the operator, and outputs an operation signal corresponding to the operation input to the processing unit 200. This function is realized by a button switch, a lever switch, a dial switch, a track pad, or a mouse, for example. In FIG. 1, the keyboard 40 corresponds to the operation input unit 120.

The display unit 130 performs various kinds of display based on the display signal from the processing unit 200. This function can be realized by a liquid crystal display (LCD) or a touch panel, for example. In FIG. 1, the video monitor 30 corresponds to the display unit 130.

The processing unit 200 performs control of the input and output of data to and from each functional unit, and calculates biological information of the subject by performing various kinds of arithmetic processing based on a predetermined program or data, the operation signal from the operation input unit 120, the reflected wave signal from the ultrasonic wave transmission and reception unit 110, and the like. This function is realized by a microprocessor, such as a central processing unit (CPU) or a graphics processing unit (GPU), or an electronic component, such as an ASIC, a field programmable gate array (FPGA), or an IC memory, for example. In FIG. 1, the main body unit 20 corresponds to the processing unit 200. In the present embodiment, the processing unit 200 includes an ultrasonic measurement control section 210, a blood vessel determination section 220, a transmission direction setting section 230, a center scanning line determination section 240, a blood vessel diameter measurement section 250, and a vascular system function information calculation section 260.

The ultrasonic measurement control section 210 includes a driving control section 211, a transmission and reception control section 212, a reception combination section 213, and a tracking section 214, and performs control relevant to the ultrasonic measurement of the ultrasonic wave transmission and reception unit 110.

The driving control section 211 generates a pulse signal having a predetermined frequency, performs transmission focusing control according to the transmission direction θ from the processing unit 200, generates a driving signal for each ultrasonic transducer, and outputs the driving signal to the transmission and reception control section 212.

The transmission and reception control section 212 generates a pulse voltage for each ultrasonic transducer according to the driving signal from the driving control section 211, and outputs the pulse voltage to the ultrasonic wave transmission and reception unit 110. In addition, an amplification or filtering process for the reflected wave signal of the ultrasonic wave from each ultrasonic transducer of the ultrasonic wave transmission and reception unit 110 is performed, A/D conversion for conversion into a digital signal is performed, and the digital signal is output to the reception combination section 213.

The tracking section 214 performs processing relevant to tracking, which is for tracking the position of a tracking region between frames of ultrasonic measurement based on the reflected wave data. For example, it is possible to perform processing for setting a tracking region in reflected wave data (for example, A-mode data) as a reference, processing for tracking each tracking region between frames, and processing for calculating the displacement of each tracking region.

The reception combination section 213 generates received data 320 by performing reception focusing control to delay the reflected wave signal from the transmission and reception control section 212 as necessary.

The received data 320 is generated for each frame. In the received data 320, an ultrasonic wave transmission direction 322, data for each scanning line 323, and B-mode data 327 are stored so as to match a frame ID 321 that is the identification number of a frame. In the data for each scanning line 323, received signal data 325 and A-mode data 326 obtained from the received signal data 325 are stored for each scanning line so as to match a scanning line ID 324 that is the identification number of a scanning line.

The B-mode data 327 is obtained from the A-mode data 326 of each scanning line.

The blood vessel determination section 220 determines the position of the blood vessel by performing ultrasonic measurement. That is, a linear scan is performed three times under the conditions in which the ultrasonic beam transmission direction is “0”, the maximum transmission direction is θmax, and the minimum transmission direction is θmin, and a total of three B-mode images generated by the respective scans are combined. A B-mode image of the maximum observation region, which is the maximum region that can be observed by the ultrasonic wave transmission and reception unit 110, is generated. Then, feature points are extracted in the B-mode image, distribution of approximately circular feature points corresponding to the cross-sectional shape of the blood vessel in the short-axis direction is determined as the position of the blood vessel wall, and the center O of the approximately circular shape is determined to be the position of the blood vessel (refer to FIGS. 3, 4A, and 4B).

The generated B-mode image of the maximum observation region is stored as composite B-mode data 330. The determined position of the blood vessel is stored as blood vessel position data 340. In addition, the maximum transmission direction θmax and the minimum transmission direction θmin are stored as transmittable range data 390.

The transmission direction setting section 230 sets the transmission direction θa of the ultrasonic beam for the measurement of the blood vessel diameter. That is, a direction θ is obtained in which an ultrasonic beam transmitted from the central ultrasonic transducer (that is, the center of the ultrasonic wave transmission unit), among the plurality of ultrasonic transducers arranged in a row in the ultrasonic wave transmission and reception unit 110, passes through the center O of the blood vessel determined by the blood vessel determination section 220. When the transmission direction θ is equal to or greater than the minimum transmission direction θmin set in the ultrasonic wave transmission and reception unit 110 and is equal to or less than the maximum transmission direction θmax, this is set as the transmission direction θ. On the other hand, the transmission direction θa is set to the minimum transmission direction θmin when the transmission direction θ is less than the minimum transmission direction θmin, and the transmission direction θa is set to the maximum transmission direction θmax when the transmission direction θ exceeds the maximum transmission direction θmax. The set transmission direction θa is stored as set transmission direction data 350.

After the transmission direction θa is set by the transmission direction setting section 230, the center scanning line determination section 240 determines a center scanning line (central transducer) that is an ultrasonic transducer (scanning line) with a transmitted ultrasonic beam passing through the center O of the blood vessel. That is, for each ultrasonic transducer 12 provided in the ultrasonic wave transmission and reception unit 110, a value obtained by totaling the signal strength difference between frames for all depth directions is further integrated over a predetermined period of time, and the ultrasonic transducer 12 having a maximum integrated value of the signal strength difference is determined to be the center scanning line (refer to FIGS. 5 and 6). The determined center scanning line is stored as center scanning line data 360.

The blood vessel diameter measurement section 250 measures a blood vessel diameter using the received data relevant to the center scanning line set by the center scanning line determination section 240. The measurement result is stored as blood vessel diameter data 370.

The vascular system function information calculation section 260 measures predetermined vascular system function information using the blood vessel diameter measured by the blood vessel diameter measurement section 250. The measurement result is stored as vascular system function information data 380.

The storage unit 300 is realized by a storage medium, such as an IC memory, a hard disk, or an optical disc, and stores various programs or various kinds of data, such as data in the operation process of the processing unit.

In addition, the connection between the processing unit 200 and the storage unit 300 is not limited to a connection using an internal bus circuit in the apparatus, and may be realized by using a communication line, such as a local area network (LAN) or the Internet. In this case, the storage unit 300 may be realized by a separate external storage device from the ultrasonic measurement apparatus 1.

In the present embodiment, the ultrasonic measurement program 310, the received data 320, the composite B-mode data 330, the blood vessel position data 340, the set transmission direction data 350, the center scanning line data 360, the blood vessel diameter data 370, the vascular system function information data 380, and the transmittable range data 390 are stored in the storage unit 300.

The ultrasonic measurement program 310 is a program for realizing the functions in the present embodiment, and each functional unit provided in the main body unit 20 is realized by executing the ultrasonic measurement program 310 in a state where the system program is being executed. The system program is a basic program for operating the main body unit 20 as a computer.

### Process Flow

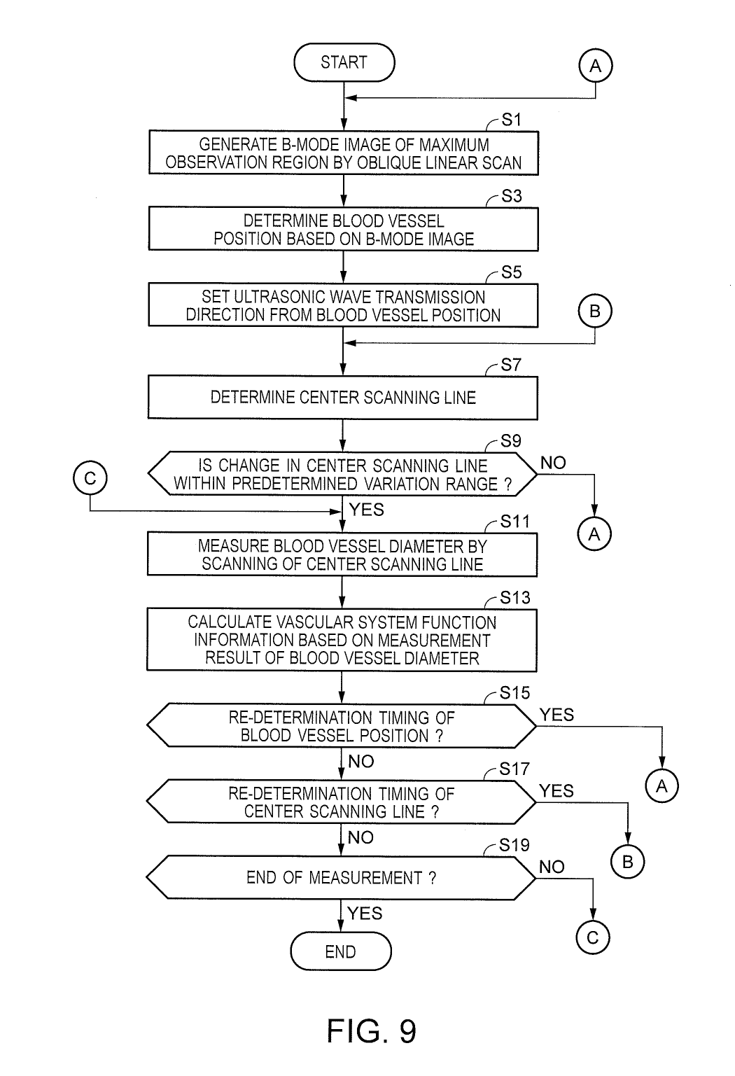

FIG. 9 is a flowchart for explaining the flow of the ultrasonic measurement process. First, the blood vessel determination section 220 generates a B-mode image of a region that can be observed by the ultrasonic wave transmission and reception unit 110 (step S1). For example, the B-mode image is generated by combining a total of three B-mode images when the transmission direction is the minimum transmission direction θmin, 0 (zero), and the maximum transmission direction θmax. Then, a blood vessel position is determined based on the generated B-mode image (step S3).

Then, based on the determined blood vessel position, the transmission direction setting section 230 sets the transmission direction θa for measuring the blood vessel diameter such that the ultrasonic beam from the ultrasonic transducer 12 located at the center of the ultrasonic transducers arranged in a row passes through the center O of the determined blood vessel position (step S5). Then, after the transmission direction θa is set, the center scanning line determination section 240 determines a center scanning line that is a scanning line (ultrasonic transducer) with a transmitted ultrasonic beam passing through the center O of the blood vessel position (step S7).

Then, it is determined whether or not a change in the determined center scanning line is within a predetermined variation range. The predetermined variation range is a range regarded as having a small change in the blood vessel position in a direction perpendicular to the transmission direction θa of the ultrasonic beam on the X-Z plane. In the predetermined variation range, for example, a difference between the number of determined center scanning lines and the number of previous center scanning lines is about several scanning lines. If the change in the center scanning line is not within the predetermined variation range (step S9: NO), it is determined that the blood vessel position has greatly changed. Therefore, the process returns to step S1 to determine the blood vessel position again.

If the change in the center scanning line is within the predetermined variation range (step S9: YES), the blood vessel diameter measurement section 250 measures a blood vessel diameter based on the received signal of the reflected wave relevant to the center scanning line (step S11). Then, the vascular system function information calculation section 260 calculates vascular system function information based on the measured blood vessel diameter (step S13).

Then, the re-determination timing of a predetermined blood vessel position is determined. If it is the re-determination timing (step S15: YES), the process returns to step S1 to determine the blood vessel position again. If it is not the re-determination timing of the blood vessel position (step S15: NO), the re-determination timing of a predetermined center scanning line is determined. If it is the re-determination timing (step S17: YES), the process returns to step S7 to determine the center scanning line again.

If it is not the re-determination timing of the center scanning line (step S17: NO), it is determined whether or not the ultrasonic measurement has ended. If the ultrasonic measurement has not ended (step S19: NO), the process returns to step S11 to measure the blood vessel diameter again. If the ultrasonic measurement has ended, this process is ended (step S19: YES).

Here, both the re-determination timing of the center scanning line and the re-determination timing of the blood vessel position assume a change in the blood vessel position in a direction perpendicular to the transmission direction θa of the ultrasonic beam on the X-Z plane. Accordingly, both the re-determination timing of the center scanning line and the re-determination timing of the blood vessel position are set as time intervals of about a few seconds to a few minutes. It is preferable that the re-determination timing of the blood vessel position is a longer time interval than the re-determination timing of the center scanning line. For example, the re-determination timing of the blood vessel position is set to a point in time when three minutes have passed immediately after last executing step S3, and the re-determination timing of the center scanning line is set to a point in time when five seconds have passed immediately after last executing step S7. Needless to say, these times are examples.

### Effects

According to the ultrasonic measurement apparatus 1 of the present embodiment, the ultrasonic measurement control section 210 generates the received data 320 by transmitting the ultrasonic wave to the blood vessel and receiving the ultrasonic wave reflected from the blood vessel. In addition, the blood vessel determination section 220 determines the blood vessel from the received data 320, the transmission direction setting section 230 sets the ultrasonic wave transmission direction for measuring the diameter of the blood vessel using the determination result of the blood vessel position, and the blood vessel diameter measurement section 250 measures the diameter of the blood vessel by transmitting the ultrasonic wave in the set transmission direction. Therefore, since it is possible to set the ultrasonic wave transmission direction suitable for the position of the blood vessel, it is possible to accurately measure the blood vessel diameter. In addition, since it is possible to accurately set the ultrasonic wave transmission method even if the blood vessel is displaced, the invention is suitable for the continuous measurement of the blood vessel diameter that follows the displacement of the blood vessel.

In addition, by setting the ultrasonic wave transmission direction to a direction passing through the center O of the blood vessel from the ultrasonic transducer located at the center of the plurality of ultrasonic transducers 12 that are provided in the ultrasonic probe 10 so as to be arranged in a row, it is possible to position the blood vessel at the approximate center in the arrangement direction of the ultrasonic transducers 12 in the observation region. Therefore, it is possible to respond to the displacement of the blood vessel in any direction. In addition, by repeating the blood vessel determination and the transmission direction setting, it is possible to measure the blood vessel diameter with the improved ability to follow the displacement of the blood vessel.

### MODIFICATION EXAMPLES

In addition, it should be understood that embodiments to which the invention can be applied are not limited to the embodiment described above and various modifications can be made without departing from the spirit and scope of the invention.

### (A) Output Intensity of Ultrasonic Wave

The output intensity of the ultrasonic wave may be changed according to the depth of the determined blood vessel. Specifically, a distance L to the center O of the blood vessel along the transmission direction θa of the ultrasonic wave is calculated, and the output intensity of the ultrasonic wave is changed so as to be proportional to the distance L. In the present embodiment, the measurement of the blood vessel diameter using the center scanning line is repeatedly performed. However, when measuring the blood vessel diameter, a displacement ΔL (=L1−L0) between a distance L1 to the center O of the blood vessel along the center scanning line and a distance L0 to the center O of the blood vessel from the center scanning line in the last measurement is calculated, and an output intensity P of the ultrasonic wave in the last measurement is increased by an intensity variation ΔP proportional to the displacement ΔL.

The entire disclosure of Japanese Patent Application No. 2014-133750 filed on Jun. 30, 2014 is expressly incorporated by reference herein.

What is claimed is:

1. A blood vessel search device, comprising:
a received data calculation unit that calculates received data by transmitting an ultrasonic wave to a blood vessel and receiving the ultrasonic wave reflected from the blood vessel;
a blood vessel determination unit that determines the blood vessel based on a calculation result of the received data calculation unit;
a transmission direction setting unit that sets a transmission direction of the ultrasonic wave for measuring a diameter of the blood vessel using a determination result of the blood vessel determination unit; and
a blood vessel diameter measurement unit that measures the diameter of the blood vessel by transmitting the ultrasonic wave in the transmission direction.

2. The blood vessel search device according to claim 1,
wherein the received data calculation unit calculates the received data by transmitting the ultrasonic wave to the blood vessel in a plurality of the transmission directions and receiving the ultrasonic wave that is reflected.

3. The blood vessel search device according to claim 1,
wherein the transmission direction setting unit sets the transmission direction such that a center of the blood vessel is included in a determination result of the blood vessel determination unit.

4. The blood vessel search device according to claim 3,
wherein the transmission direction setting unit sets the transmission direction to a direction passing through the center of the blood vessel from a part of a transmission unit of the ultrasonic wave.

5. An ultrasonic measurement apparatus, comprising:
the blood vessel search device according to claim 1; and
a blood vessel information measurement unit that measures information of the blood vessel using the calculation result of the received data calculation unit in the transmission direction set by the transmission direction setting unit.

6. An ultrasonic measurement apparatus, comprising:
the blood vessel search device according to claim 2; and
a blood vessel information measurement unit that measures information of the blood vessel using the calculation result of the received data calculation unit in the transmission direction set by the transmission direction setting unit.

7. An ultrasonic measurement apparatus, comprising:
the blood vessel search device according to claim 3; and
a blood vessel information measurement unit that measures information of the blood vessel using the calculation result of the received data calculation unit in the transmission direction set by the transmission direction setting unit.

8. An ultrasonic measurement apparatus, comprising:
the blood vessel search device according to claim 4; and
a blood vessel information measurement unit that measures information of the blood vessel using the calculation result of the received data calculation unit in the transmission direction set by the transmission direction setting unit.

9. The ultrasonic measurement apparatus according to claim 5,
wherein the blood vessel information measurement unit measures a diameter of the blood vessel using the calculation result in a transmission direction passing through a center of the blood vessel.

10. The ultrasonic measurement apparatus according to claim 5,
wherein measurement of information of the blood vessel by the blood vessel information measurement unit is continued by executing the blood vessel determination of the blood vessel determination unit and the transmission direction setting of the transmission direction setting unit so as to follow displacement of the blood vessel.

11. The ultrasonic measurement apparatus according to claim 5, further comprising:
an ultrasonic wave output change unit that changes an output intensity of the ultrasonic wave according to a distance change from the transmission unit to the blood vessel due to displacement of the blood vessel.

12. A blood vessel search method, comprising:
calculating received data by transmitting an ultrasonic wave to a blood vessel and receiving the ultrasonic wave reflected from the blood vessel;
determining the blood vessel based on a calculation result of the received data; and
setting a transmission direction of the ultrasonic wave for measuring information of the blood vessel using the determination result.

13. An ultrasonic measurement apparatus, comprising:
an ultrasonic wave transmission and reception unit;
a blood vessel determination unit that determines a blood vessel based on information obtained from a linear scan in a first transmission direction by the ultrasonic wave transmission and reception unit; and
a transmission direction setting unit that sets the first transmission direction to a transmission direction passing through a center position of the blood vessel obtained by the blood vessel determination unit.

14. The ultrasonic measurement apparatus according to claim 10,
wherein the blood vessel determination unit determines the blood vessel based on the first transmission direction and information obtained by linear scans in second and third transmission directions, the first transmission direction being positioned between the second and third transmission directions.
